(19) United States
(12) Patent Application Publication
(10) Pub. No.: US 2010/0204526 A1
(43) Pub. Date: Aug. 12, 2010

(54) PROCESS FOR SOLVENT PRODUCTION UTILIZING LIQUID PHASE ADSORPTION

(75) Inventors: Jay Kouba, Chicago, IL (US); Anil Oroskar, Oak Brook, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: TETRAVITAE BIOSCIENCE, INC., Chicago, IL (US)

(21) Appl. No.: 12/677,736

(22) PCT Filed: Sep. 10, 2008
(86) PCT No.: PCT/US2008/075873

§ 371 Date: Mar. 11, 2010

Related U.S. Application Data
(60) Provisional application No. 60/993,348, filed on Sep. 11, 2007.

(51) Int. Cl. C07C 29/74

(52) U.S. Cl. 568/840

(57) ABSTRACT

Methods and systems are provided for the separation of solvents, including, but not limited to, butanol, from a fermentative solventogenesis reaction medium that utilizes Clostridium beijerinckii NCIMB 8052 or derivatives thereof, including, but not limited to, Clostridium beijerinckii BA101, ATCC No. PTA-1550, by contacting the reaction medium directly with an adsorbent that selectively adsorbs the solvent; separating the adsorbent/solvent adsorbate from the reaction medium; and desorbing the solvent adsorbate from the adsorbent.

### CROSS-REFERENCE TO RELATED APPLICATIONS

This patent application claims the benefit of U.S. Provisional Patent Application No. 60/993,348, filed Sep. 11, 2007, which is incorporated by reference.

### FIELD OF INVENTION

The compositions and methods described herein pertain to the separation of solvents, including, but not limited to, the adsorptive separation of butanol from a fermentative solventogenesis medium.

### BACKGROUND OF THE INVENTION

With increased energy security issues, heightened concerns regarding climate change, and global depletion of petroleum reserves there has been an escalating worldwide interest in renewable energies. There is a growing consensus that producing liquid biofuels such as ethanol and butanol from renewable and inexpensive plant materials has a great potential to meet a large portion of this nation's energy demand in the transportation sector. The expanding market for biofuels also simultaneously addresses the issues of energy supply and lower greenhouse gas emissions. Two federal policies are further motivating greater use of biofuels: a $0.51 tax credit per gallon of ethanol used as motor fuel and a mandate for up to 7.5 billion gallons of “renewable fuel” to be used in gasoline by 2012, the latter included in the Energy Policy Act (EPACT 2005).

Similar to ethanol, butanol has many favorable attributes as a fuel molecule. However, it is an underexploited biofuel. Butanol can be produced as a co-product with ethanol and acetone from carbohydrates through fermentation by several solventogenic Clostridia. Compared to the currently popular fuel additive ethanol, butanol has several advantages. It contains around 22% oxygen which when used as a fuel will result in more complete combustion and lower exhaust smoke. In addition, it has a higher energy content (BTU/volume) than ethanol, is more miscible with gasoline and diesel, and has lower vapor pressure and solubility characteristics which would allow it to be shipped by pipeline, unlike ethanol.

The separation of ethanol, butanol, and other solvents from the fermentation process is a critical aspect to the overall energy efficiency and financial feasibility of producing such biofuels. This is particularly true because biofuel solvents are toxic to most cells and organisms producing the solvents. Thus separation processes that can limit the potential concentration of biofuel solvents in the fermentation or reaction medium are needed.

### BRIEF SUMMARY OF THE INVENTION

Described herein are methods and systems for the separation of solvents, including, but not limited to, butanol, from a fermentative solventogenesis reaction medium that utilizes Clostridium beijerinckii NCIMB 8052 or derivatives thereof, including, but not limited to, Clostridium beijerinckii BA101, ATCC No. PTA-1550, by contacting the reaction medium directly with an adsorbent that selectively adsorbs the solvent; separating the adsorbent/solvent adsorbate from the reaction medium; and desorbing the solvent adsorbate from the adsorbent.

### BRIEF DESCRIPTION OF THE DRAWINGS

FIG. 1 depicts a graph of solvent concentration vs. time for Clostridium beijerinckii BA101 fermentation with carbon adsorbent addition.

FIG. 2 depicts a graph of acid concentration vs. time for Clostridium beijerinckii BA101 fermentation with carbon adsorbent addition.

FIG. 3 depicts a graph of solvent concentration vs. time for Clostridium beijerinckii BA101 fermentation with XAD 4, C18, Zeolite, and Orpheus Silicalite adsorbent addition.

FIG. 4 depicts a graph of acid concentration vs. time for Clostridium beijerinckii BA101 fermentation with XAD 4, C18, Zeolite, and Orpheus Silicalite adsorbent addition.

FIG. 5 depicts a graph of Clostridium beijerinckii BA101 concentration vs. time and Glucose concentration vs. time for solvent recovery from fermentation broth via a continuous expanded-bed adsorption process.

### DETAILED DESCRIPTION OF THE INVENTION

Described herein are: 1) Fermentative Solventogenesis Processes, 2) Reaction Medium Compositions, 3) Methods of Separating Solvents from a Reaction Medium, 4) Process Control for Use in the Methods Described Herein, 5) Adsorbents for Use in the Methods Described Herein, and 6) Desorbents for Use in the Methods Described Herein.

In order to provide a more thorough understanding of the present invention, the following description sets forth numerous specific details, such as specific methods, parameters, examples, and the like. It should be recognized, however, that such description is not intended as a limitation on the scope of the present invention, but is intended to provide a better understanding of the exemplary embodiments. Unless otherwise specified, all percentages specifying concentrations are in weigh per volume (w/v) and specifically in grams per liter (g/L).

All patents, scientific articles, and other publications recited in this specification are hereby incorporated by reference in their entirety for all purposes.

Fermentative Solventogenesis Processes

In the broadest sense, any reaction using Clostridium beijerinckii NCIMB 8052 or any derivative thereof, or generational (e.g. second, third, fourth, etc generation) derivative of such derivative, including, but not limited to, Clostridium beijerinckii BA101, ATCC No. PTA-1550, can be used in the methods described herein. Such derivatives can be created via natural selection, chemical or radiation induced mutation, importation of other biosynthetic pathways (or engineering of the existing pathway), or any other mutation or genetic modification means.

The fermentation of carbohydrates to acetone, butanol, and ethanol (ABE) by solventogenic microorganisms including Clostridia is known. U.S. Pat. No. 6,358,717 describes production of solvents using a mutant strain of Clostridium beijerinckii, designated Clostridium beijerinckii BA101, which is incorporated herein by reference in its entirety.

One problem associated with ABE fermentation by Clostridium beijerinckii and other solventogenic microorganisms is solvent toxicity to the culture. One method to overcome this is continuous removal of the toxic solvents during the process for maximum production of solvents.

Some efforts have been made to improve the Clostridia-based butanol fermentation processes by developing new strains and downstream technologies. For example, as described in U.S. Pat. No. 6,358,717, which is incorporated herein by reference in its entirety, Blaschek and others used chemical mutagenesis to develop a mutant strain of Clostridium beijerinckii, BA101 with higher butanol concentration. To circumvent butanol inhibition, Blaschek and others also developed various downstream processes including gas stripping, pervaporation, and liquid-liquid extraction. See, e.g., Ezeji, T. C., Qureshi, N. & Blaschek, H. P. Butanol fermentation research: Upstream and downstream manipulations. Chem Rec 4, 305-314 (2004); US Pat. Pub. No. 2005/0089979; Qureshi et al., Butanol production using Clostridium beijerinckii BA101 hyper-butanol producing mutant strain and recovery by pervaporation, Appl Biochem Biotech 84-6, 225-235 (2000); and Ezeji et al., Acetone butanol ethanol (ABE) production from concentrated substrate: reduction in substrate inhibition by fed-batch technique and product inhibition by gas stripping, Appl Microbiol Biot 63, 653-658 (2004), each of which is incorporated herein by reference in its entirety.

Reaction Medium Composition

In the broadest sense, any combination of substrate and Clostridium beijerinckii NCIMB 8052 or derivatives thereof (as discussed above), including, but not limited to, Clostridium beijerinckii BA101, ATCC No. PTA-1550, which is capable of producing solvents, can be used in the methods described herein.

In some variations, the reaction medium includes butanol in concentrations including: 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%, as well as ranges defined by any two of the aforementioned values.

In some variations, the reaction medium includes butanol in concentrations between 0.5% and 0.7% (e.g., between 0.55% and 0.65%).

In some variations, the reaction medium includes solvents in concentrations greater than 0.1% and less than 12%, such as 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, or 11.5%, as well as ranges defined by any two of the aforementioned values (e.g., 0.5% to 4%).

In some variations, the reaction medium solvents include a mixture of acetone, butanol, and ethanol, a mixture of acetone and butanol, or any other combination of butanol, ethanol, and/or acetone.

In the broadest sense, any substrate that contains any amount of fermentable sugar can be used in the methods described herein.

In some variations, the reaction medium includes a substrate in the form of glucose, pentose, starch, liquefied starch, enzyme-treated liquefied starch, maltodextrin, and corn steep liquor.

In some variations, cellulosic and hemicellulosic materials can be converted to downstream products such as fermentable sugars by various methods. In some variations, biomass, lignocellulosic, or cellulosic materials are converted to downstream products such as fermentable sugars via a method which does not require living bacteria, yeast, or other organisms. In some variations, biomass, lignocellulosic, or cellulosic materials are converted to downstream products such as fermentable sugars via a method which utilizes living bacteria, yeast, or other organisms.

In the broadest sense, any additive that assists in the fermentation of the substrate into solvents can be used in the methods described herein. In some variations, additives include Tryptone Glucose Yeast extract (TGY), salts, buffers, vitamins, minerals, and/or yeast.

In some variations, the solventogenic organism can include Clostridium beijerinckii NCIMB 8052 or derivatives thereof (as discussed above), including, but not limited to, the Clostridium beijerinckii BA101, ATCC No. PTA-1550, mutant as described in U.S. Pat. No. 6,358,717, which is incorporated herein by reference in its entirety.

Methods of Separating Solvents from the Reaction Medium

In the broadest sense, any adsorption process that is capable of separating solvents from a reaction medium can be used in the methods described herein.

In some variations, an adsorption separation process includes an adsorption unit including at least one adsorbent bed, a multi-stage adsorption unit comprising a plurality of adsorption stages or adsorption vessels, a multi-bed adsorption unit comprising a plurality of adsorption beds, or any combination thereof. The solvent separation can take place in any convenient mode, for example, a fixed bed, a fluidized bed, an expanded bed, a moving bed, a swing bed, a simulated moving bed, or any combination thereof, depending on the type of process desired. These types of reactors and their designs are described in “Perry's Chemical Engineers' Handbook,” Eds. R. H. Perry, D. W. Green and J. O. Maloney, McGraw-Hill Book Company, 6th ed., 1984, which is hereby incorporated by reference.

In some variations, the separation process can include adsorption integrated inside the fermentor. In some variations, the separation process can include adsorption outside the fermentor. In some variations, the separation process can include adsorption being contacted with the reaction medium in the fermentor and the solvent being desorbed outside of the fermentor.

A separation process which includes a fixed bed adsorption column for separation of a fermentation reaction typically includes filtration and/or centrifugation in order to remove components of the reaction medium (i.e., the organisms) before the mixture is applied to the fixed bed. The filtration and/or centrifugation process helps to avoid clogging of the solid-phase bed resulting in increased back pressures, which might disturb the flow through the bed.

In some variations, the separation process can include a fluidized, expanded, or moving bed process. By using a fluidized, expanded, or moving process, it is possible to avoid the above-mentioned filtration and/or centrifugation operational steps before application of the raw material to the column, due to the greater ease of particles passing through the bed and column. Thus, time and expenses for these processes are reduced. A non-limiting example of a fluidized or expanded bed includes a process where the solid phase particles (adsorbents) are kept in a free, fluid phase by applying a flow having an opposite direction to the direction of the relative movement of the solid phase particles. In some variations, the separation process can include a fluidized, expanded, or moving bed process, in addition to an organism filtration process or an organism anchoring design.

In some variations, the expanded bed process includes one or more up-flow fluid reactors that have the reaction medium inlet at or near the bottom of the reactor when the adsorbent has a relative density larger than that of the reaction medium. In some variations, the expanded bed process includes one or more down-flow fluid bed reactors that have the reaction medium inlet at or near the top of the reactor when the adsorbent has a relative density less than that of the reaction medium.

A non-limiting example of an expanded bed up-flow process includes: First, an adequate quantity of adsorbent is placed in a column. Second, fluid flow through the adsorbent from below is initiated by pumping the reaction medium through a fluid distributor. The adsorbent is thereby fluidized (expanded). Third, the adsorbent is rinsed in the column and the conductivity (i.e., salt concentration) and pH are adjusted to what is required to allow binding of the solvents to the adsorbent. Fourth, the reaction medium is applied to the expanded bed of adsorbents and the solvents are bound. Fifth, the remaining reaction medium can be rinsed out from the column using a wash fluid. Sixth, the solvents are desorbed off the adsorbent medium by applying a desorbent that weakens the interaction with the adsorbent. The desorption of the solvent can be performed after packing the adsorbent by reversing the flow direction in the column, or the desorption can be performed in the expanded bed state. Seventh, the adsorbent can be optionally rinsed and regenerated.

In some variations, any of the foregoing separation processes could include a swing-bed system. A non-limiting example of a swing-bed system includes a set of two or more beds of adsorbent that can be employed with appropriate valving so that the reaction medium can be passed through one or more adsorbent beds of a set while a desorbent material can be passed through one or more of the other beds in a set. The flow of a feed mixture and a desorbent material can be either up or down through an adsorbent in such beds.

In some variations, the fluidized bed should be free of bubbles, be homogeneous, maintain particle suspension and manifest noncritical flow velocity control for various bed heights and bed densities. In some variations, the process includes procedures and systems to effect the foregoing fluidized bed characteristics, for example, by the use of baffles, packing, mechanical vibration, and mixing devices, the use of mixed particle sizes, special flow control valves, bed rotation, etc.

In some variations, certain improvements in fluidized beds can be effected by externally applying a magnetic field to a fluidized bed of particulate solids having ferromagnetic properties, as described in U.S. Pat. Nos. 3,304,249; 3,440,731; and 3,439,899, each of which is incorporated herein by reference in its entirety.

In some variations, the process can include methods for the prevention of bubble formation in fluidized beds by using an externally applied magnetic field in conjunction with a bed of permanent magnets as described in U.S. Pat. No. 3,439,899, which is incorporated herein by reference in its entirety. U.S. Pat. No. 3,439,899 also disclosed utilizing alternating current to provide an electromagnetic field to this fluidized bed process.

In some variations, the processes can utilize gradient applied magnetic fields to generate body forces to hold finer adsorbents in place and thus permit higher flow rates than in conventional fluidized beds as described in British Pat. No. 1,148,513, which is incorporated herein by reference in its entirety.

In some variations, the external magnetic field can be provided by either a permanent magnet or electromagnet coaxially surrounding the bed and connected to a power source to produce the desired current.

In some variations, the separation process can include a moving bed adsorption process. Moving bed systems can have much greater separation efficiency than fixed bed systems. In some variations, the moving bed process has retention and displacement/desorbent operations that are continuously taking place which allows both continuous production of an extract and a raffinate stream and the continual use of reaction medium and displacement/desorbent fluid streams. In some variations of the moving bed adsorption process, the adsorbent circulates continuously as a dense bed in a closed cycle and moves up (or down) the adsorbent chamber from bottom to top (or from top to bottom). Liquid streams flow down (or up) through the bed counter-currently to the solid.

In some variations, the adsorption and displacement/desorption can be integrated in one unit. In some variations, the adsorption and displacement/desorption take place in separate units. In some variations, in the process that includes adsorption and displacement/desorption in separate units, the adsorbent/adsorbate can be washed, and any remaining reaction medium can be recycled to the fermentor.

A non-limiting example of a process wherein the adsorption and displacement/desorption can be integrated in one unit includes a moving bed unit, separate from the fermentor. In some variations, the reaction medium can be introduced at any point in the moving bed unit, including below the desorbent input. In some variations, the desorbent can be introduced to the bed at a higher or lower level. The desorbent is a liquid of a different boiling point from the reaction medium and the solvents, and can displace the reaction medium and the solvents from the adsorbent. Conversely, the reaction medium and the solvents can displace the desorbent from the adsorbent with proper adjustment of relative flow rates of solid and liquid. The reaction medium with the solvent removed is withdrawn from a position below the feed entry. Only a portion of the liquid flowing in the bed is withdrawn at this point; the remainder continues to flow into the next section of the bed. The solvent product, consisting of the solvent and desorbent, is withdrawn from the bed at a point higher than the feed. Again, only a portion of the flowing liquid in the bed is withdrawn, and the remainder continues to flow into the next bed section.

In some variations, the separation process can include a simulated moving bed countercurrent flow system. A non-limiting example of such a system includes the progressive movement of multiple liquid access points down an adsorbent chamber that simulates the upward movement of the adsorbent contained in the chamber, such as described in U.S. Pat. No. 2,985,589 and U.S. Pat. No. 4,940,830, which are incorporated herein by reference. Cyclic advancement of the input and output streams can be accomplished by a manifolding system, which are also known, e.g., by rotary disc valves shown in U.S. Pat. No. 3,040,777 and U.S. Pat. No. 3,422,848, which are incorporated herein by reference. Equipment utilizing these principles is known, in sizes ranging from pilot plant scale (U.S. Pat. No. 3,706,812) to commercial scale.

In some variations, the solvents can be purified and further separated subsequent to being separated from the adsorbent in a standard series of distillation columns. These well-known separation techniques and their designs are described in “Perry's Chemical Engineers' Handbook,” Eds. R. H. Perry, D. W. Green and J. O. Maloney, McGraw-Hill Book Company, 6^{th} ed., 1984, which is hereby incorporated by reference.

Process Control for Use in the Methods Described Herein

In the broadest sense, any process control methodology and ranges for control variables that allow the methods described herein to separate solvents from a reaction medium can be used in the methods described herein. In some variations, the process control will maintain specific activity of fermentation (i.e., rate of consumption of the substrate, purity and recovery of solvents from the reaction medium) and prevent any external contamination (i.e., oxygen) which could cause irreversible deactivation of the bacterial culture.

In some variations, the feed rate of the separation medium will be governed by the concentration of solvents in the reaction medium. Additionally, the density, viscosity, and velocity of the reaction medium and the diameter and density of the adsorbent will affect the balancing of frictional versus gravitational forces.

In some variations, the temperature of the fermentor will be 37° C. In some variations, the temperature of the fermentor will be between 27° C. and 37° C. In some variations, the temperature of the fermentor will be between 37° C. and 47° C. In some variations, the temperature of the fermentor will be between 32° C. and 42° C. In some variations, the temperature of the fermentor will be between 27° C. and 47° C. (e.g., about 30° C., 32° C., 34° C., 36° C., 38° C., 40° C., 42° C., 44° C., or 46° C., as well as ranges defined by any two of the aforementioned values).

In some variations, the temperature of the regeneration/desorbent unit will be 90° C. In some variations, the temperature of the regeneration/desorbent unit will be between 70° C. and 90° C. In some variations, the temperature of the regeneration/desorbent unit will be between 90° C. and 160° C. In some variations, the temperature of the regeneration/desorbent unit will be between 70° C. and 150° C. In some variations, the temperature of the regeneration/desorbent unit will be between 70° C. and 160° C. (e.g., 75° C., 80° C., 85° C., 90° C., 95° C., 100° C., 105° C., 110° C., 115° C., 120° C., 125° C., 130° C., 135° C., 140° C., 145° C., 150° C., or 155° C., as well as ranges defined by any two of the aforementioned values).

In some variations, the pressure of the fermentor will be 550 mmHg. In some variations, the pressure of the fermentor will be between 450 mmHg and 650 mmHg (e.g., 475 mmHg, 500 mmHg, 525 mmHg, 550 mmHg, 575 mmHg, 600 mmHg, or 625 mmHg, as well as ranges defined by any two of the aforementioned values). In some variations, the pressure of the fermentor will be between 500 mmHg and 600 mmHg.

In some variations, the pressure of the fermentor will be at least 0.1 atm and less than 5 atm (e.g., 1 atm, 2 atm, 3 atm, or 4 atm, as well as ranges defined by any two of the aforementioned values).

In some variations, the pH of the fermentor contents will be 4.8. In some variations, the pH of the fermentor contents will between 4.6 and 5. In some variations, the pH of the fermentor contents will be between 4.5 and 6.5. In some variations, the pH of the fermentor contents will between be 4 and 7 (e.g., 4.2, 4.4, 4.6, 4.8, 5, 5.2, 5.4, 5.6, 5.8, 6, 6.2, 6.4, 6.6, or 6.8, as well as ranges defined by any two of the aforementioned values). In some variations, higher pH will decrease the adsorption of fermentation intermediates such as acetic and butyric acids.

In some variations, the process control for a simulated moving bed system can be guided by the methods and procedures described in U.S. Pat. No. 3,268,604, U.S. Pat. No. 3,268,603, U.S. Pat. No. 3,131,232, U.S. Pat. No. 5,912,395, U.S. Pat. No. 5,470,482, U.S. Pat. No. 5,457,260, U.S. Pat. No. 6,284,134, U.S. Pat. No. 6,096,218, and U.S. Pat. No. 5,569,808, which are incorporated herein by reference.

Adsorbents for Use in the Methods Described Herein

In the broadest sense, any adsorption that is capable of selectively adsorbing solvents from a reaction medium can be used in the methods described herein. The functions and properties of adsorbents in the chromatographic separation of liquid components are well-known (e.g., U.S. Pat. No. 4,642,397, U.S. Pat. No. 3,133,126, U.S. Pat. No. 3,843,518, U.S. Pat. No. 3,686,343, U.S. Pat. No. 3,724,170, U.S. Pat. No. 3,626,020, U.S. Pat. No. 3,558,730, U.S. Pat. No. 3,558,732, U.S. Pat. No. 3,663,638, U.S. Pat. No. 3,686,342, U.S. Pat. No. 3,734,974, U.S. Pat. No. 3,706,813, U.S. Pat. No. 3,851,006, U.S. Pat. No. 3,698,157, U.S. Pat. No. 3,917,734, U.S. Pat. No. 3,665,046, U.S. Pat. No. 3,510,423, U.S. Pat. No. 3,723,561, U.S. Pat. No. 3,851,006, and U.S. Pat. No. 3,929,669, which are incorporated herein by reference).

In selection of an adsorbent, the adsorbent's capacity for adsorbing a specific volume of one or more extract components is considered. In some variations, the higher the adsorbent's capacity for an extract component, the lesser is the amount needed of such adsorbent to separate the extract component for a particular rate of feed mixture. A reduction in the amount of adsorbent required for a specific adsorptive separation can reduce the cost of the separation process. In addition to the initial capacity of the adsorbent, the sustainability of the capacity during actual use in the separation process over the life of the adsorbent is also considered.

In some variations, the adsorbent has the capacity to adsorb butanol in concentrations including: 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2.0%, as well as ranges defined by any two of the aforementioned values (e.g., 0.4% to 1.6%).

In some variations, the adsorbent has the capacity to adsorb solvents in concentrations including: 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, or 12%, as well as ranges defined by any two of the aforementioned values (e.g., 4% to 8%).

In some variations, the adsorbent possesses adsorptive selectivity for butanol or the solvents as compared to the other components of the reaction medium, including, but not limited to, the any nutrients, substrates, additives, organisms, and reaction intermediates (acetic acid and butyric acid, etc). Relative selectivity can be expressed not only for one feed component as compared to another, but can also be expressed between any feed mixture component and the desorbent material.

In some variations, the adsorbent is not toxic to the organism. In some variations, the adsorbent will not stop the fermentation process. A person of ordinary skill in the art can test for adsorbent toxicity in a manner described in the examples below or in any other method known in the art.

In some variations, the adsorbent includes hydrophobic adsorbents (e.g., C18) with high selectivity over water. Such hydrophobic characteristics can reduce downstream purification costs.

In some variations, the adsorbent has an advantageous rate of desorption of the extract component. This characteristic can relate to the amount of desorbent material that must be employed (or amount of heat that must be employed) in the process to recover the extract component from the adsorbent. Faster rates of desorption can reduce the amount of desorbent material needed to remove the extract component and, therefore, permit a reduction in the operating cost of the process. With faster rates of desorption, less desorbent material has to be pumped through the process and, in some variations, separated from the extract stream for reuse in the process.

In some variations, the adsorbent has a spherical geometry to assist in durability and proper hydrodynamic flow in moving bed processes. In some variations, the physical dimensions of the adsorbent will allow quick settling after an adsorption cycle in preparation for the desorption cycle.

In some variations, the adsorbent includes inorganic materials. Non-limiting examples of inorganic adsorbent materials include, but are not limited to silica, bonded silica (C18), end capped silica, silica gels, silica macroscopic rods, silicalite, alumina, activated alumina, and functionalized alumina.

In some variations, the adsorbent includes crystalline inorganic materials. Non-limiting examples of crystalline inorganic materials include, but are not limited, to zeolites and various cation exchanged zeolites.

In some variations, the adsorbent includes organic materials. Non-limiting examples of organic materials include, but are not limited to, carbon, activated carbon, Calgon OL, and polymeric materials (including, but not limited to, XAD4, Polystyrene-DVB, and methacrylates).

In some variations, the adsorbent includes ion exchanges/molecular sieves including, but not limited to, carbon molecular sieves, ion exchange resins, zeolites, montmorillonite, clay, and soil humus.

Desorbents for Use in the Methods Described Herein

The functions and properties of desorbents in the chromatographic separation of liquid components are well known (e.g., U.S. Pat. No. 4,642,397, U.S. Pat. No. 3,133,126, U.S. Pat. No. 3,843,518, U.S. Pat. No. 3,686,343, U.S. Pat. No. 3,724,170, U.S. Pat. No. 3,626,020, U.S. Pat. No. 3,558,730, U.S. Pat. No. 3,558,732, U.S. Pat. No. 3,663,638, U.S. Pat. No. 3,686,342, U.S. Pat. No. 3,734,974, U.S. Pat. No. 3,706,813, U.S. Pat. No. 3,851,006, U.S. Pat. No. 3,698,157, U.S. Pat. No. 3,917,734, U.S. Pat. No. 3,665,046, U.S. Pat. No. 3,510,423, U.S. Pat. No. 3,723,561, U.S. Pat. No. 3,851,006, and U.S. Pat. No. 3,929,669, which are incorporated herein by reference).

In some variations, the desorbent includes materials that are substances capable of removing a selectively adsorbed feed component from the adsorbent. In some variations, the desorbent includes materials that displace the extract components from the adsorbent with reasonable mass flow rates without the desorbant being so strongly adsorbed as to unduly prevent the extract component from displacing the desorbent material in a following adsorption cycle. Expressed in terms of the selectivity, the adsorbent is more selective for the extract component with respect to a raffinate component than it is for the desorbent material with respect to a raffinate component.

In some variations, the desorbent includes materials that are compatible with the particular adsorbent and the particular feed mixture. More specifically, they must not reduce or destroy the critical selectivity of the adsorbent for the extract components.

In some variations, desorbent materials include substances which are easily separable from the feed mixture that is passed into the process. In some variations, after desorbing the extract components of the feed, both desorbent materials and the extract components are typically removed in admixture from the adsorbent. In some variations, one or more raffinate components are typically withdrawn from the adsorbent in admixture with desorbent materials and without a method of separating at least a portion of the desorbent materials, such as distillation; neither the purity of the extract product nor the purity of the raffinate product will be very high. In some variations, the desorbent materials used in the separation process will have a substantially different average boiling point than that of the feed mixture to allow separation of desorbent materials from feed components in the extract and raffinate streams by simple fractionation, thereby permitting reuse of desorbent materials in the process.

In some variations, the solvent adsorbate can be separated from the adsorbent through a process including, but not limited to, heat treatment or pressure swing. In some variations, the solvent adsorbate can be separated from the adsorbent with desorbents including, but not limited to, hot water, steam, hot gases, hot air, a hot carbon dioxide and hydrogen mixture, supercritical carbon dioxide, or other solvents, such as methanol. In some variations, the desorbents include a pressure swing system.

It is understood in the art that cycle times for swing bed systems will vary depending on the desorbent utilized. In some variations, the cycle time for a hot water desorbent system can vary from ten to twenty minutes (twelve, fourteen, sixteen, or eighteen minutes). In some variations, the cycle time for a hot air desorbent system can vary from six to eight hours (e.g., seven hours).

In some variations, the desorption step will include a thermal process that is facilitated with carbon dioxide, which allow for lower desorption temperatures, faster cycle times, reduced adsorbent inventory, and improve energy efficiency. Carbon dioxide is a byproduct of the fermentation process and is thus readily available as a desorbent. Carbon dioxide is also a suitable desorbent because of favorable affinities for activated carbon, silicalite, or other hydrophobic materials. This allows lower temperature of desorption which in turn reduces cycle time of the desorption cycle and the adsorbent inventory in the system.

### Examples

### Example 1

### BA101 Fermentation with Calgon OL Adsorbent Addition

### Organism, Culture Maintenance, and Fermentation Conditions

C. beijerinckii BA 101 was used for these studies. Spores (200 μl) were heat shocked for 10 min. at 80° C. followed by cooling in an anaerobic chamber for 5 min. The culture was inoculated into 10 ml Tryptone-glucose-yeast extract (TGY) medium (in 50 ml screw capped pyrex bottle) and was incubated anaerobically for 12-14 h at 36±1° C.

The composition of the TGY media is as follows: Tryptone (30 g/L), Glucose (20 g/L), and Yeast extract (10 g/L). Other nutrient media can be used. Useful nutrient media include those known to the art, such as P2. The nutrient media optionally can contain additives such as salts.

The composition of P2 media is as follows: Glucose (60-100 g/L) and Yeast extract (1-1.5 g/L). On cooling to 35° C. under oxygen-free nitrogen atmosphere, filter-sterilized P2 stock solutions [(buffer: KH₂PO₄, 50 gL⁻¹; K₂HPO₄, 50 gL⁻¹; Ammonium acetate, 220 gL⁻¹), (vitamin: Para-amino-benzoic acid, 0.1 gL⁻¹; Thiamin, 0.1 gL⁻¹; Biotin, 0.001 gL⁻¹), (mineral: MgSO₄.7H₂O, 20 gL⁻¹; MnSO₄.H₂O, 1 gL⁻¹; FeSO₄.7H₂0, 1 gL⁻¹; NaCl, 1 gL⁻¹)] were added.

### Batch Fermentation

These experiments were done in 100 mL batches in a 160 mL milk dilution bottle, placed inside an anaerobic chamber in an incubator maintained at 35° C. This media was prepared, autoclaved, and equilibrated in anaerobic conditions prior to inoculation. These bottles (batch reactors) were inoculated with the 10 mL □noculums of TGY extract with a large number of C. beijerinckii BA101 prepared as described in the previous paragraphs.

### Analysis of Fermentation

Glucose concentration was determined using a hexokinase and glucose-6-phosphate dehydrogenase (Sigma Chemicals, St. Louis, Mo., USA) coupled enzymatic assay. The analysis of the media was performed for the ABE and acids concentration using the GC analysis. The total amount of ABE produced and acids (acetic and butyric) were measured using a 6890 Hewlett-Packard gas chromatograph (Hewlett-Packard, Avondale, Pa.) equipped with a flame Ionization detector (FID) and 6 ft×2 mm glass column (10% CW-20M, 0.01% H₃PO₄, support 80/100 Chromosorb WAW). The measurement procedure was as follows:

Preparation of Acetone-Butanol-Ethanol standard: A) Standard solutions of acetone, butanol, and ethanol were prepared with distilled water (acetone 2 g/L, butanol 5 g/L, and ethanol 2 g/L). B) A standard solution (50 g/L) of internal standard (n-propanol) was prepared with distilled water. 1 ml of A and 0.1 ml of B were mixed. 1 μL of the mixture was injected into GC and the peak areas of acetone, butanol, ethanol, and n-propanol were shown in the chromatogram. The order of the peaks is acetone, ethanol, n-propanol, butanol, Acetic acid, and Butyric acid.

Preparation of samples for GC analysis: Aliquots of samples were taken from the fermentor and centrifuged at 14,000 rpm for 3 min at 4° C. 25 μL of the internal standard was added to 250 μL of the supernatant and mixed. 1 μL of the mixture was injected into GC and the chromatogram displayed the individual ABE peak areas. The concentration of the acetone, butanol, or ethanol is calculated as follows:

From the peak areas, Response Factors (RF) for each peak was calculated as follows: acetone (RF)=(internal standard peak area/acetone peak area)/(wt of internal standard(5 g)/acetone wt(2 g)) Conc. in g/L(acetone)=(Wt of internal standard(5 g)×RF×Peak area of acetone)/(Peak area of internal standard)

### Adsorbent Addition

The Calgon OL adsorbent was added after about 18 hrs of fermentation to the 100 mL P2 medium. The adsorbent addition was done in 3 batches of 2 g every two hours (i.e, at 18, 20, and 24 hrs.) and one batch of 6 g 6 hrs. from then (i.e, at 30, 36, and 42 hrs). The fermentation was deemed to be complete after about 72 hours. Trial 1 was a control trial in which no adsorbent was added. In trial 2 and trial 3 Calgon OL adsorbent was added as discussed above.

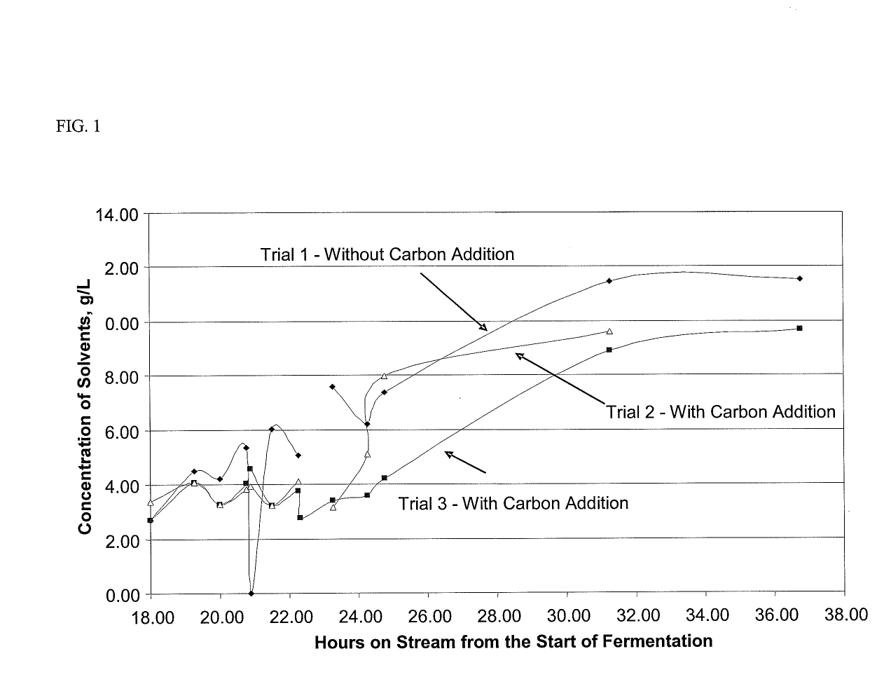

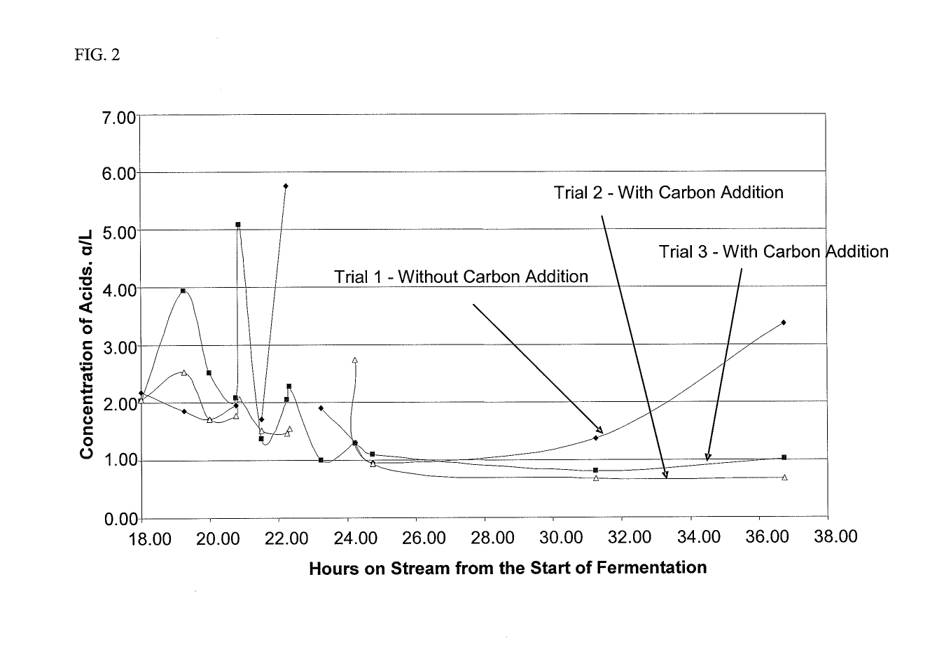

The concentration of acetone, butanol, and ethanol (ABE) in g/L and the concentration of intermediate acids (such as acetic and butyric acids) in g/L were analyzed as discussed above. The results are shown in FIG. 1 and FIG. 2.

Referring to FIG. 1 and FIG. 2, in both Trial 2 and Trial 3 the concentration of ABE was reduced subsequent to each addition of the Calgon OL adsorbent. This suggests that Calgon OL can successfully adsorb ABE from the reaction medium under these solventogenic fermentation conditions.

Referring again to FIG. 1 and FIG. 2, the solventogenic fermentation continued subsequent to the addition of the Calgon OL adsorbent in both Trial 2 and Trial 3, which furthers the proposition that the Calgon OL adsorbent is not toxic to Clostridium beijerinckii BA101.

Thus, Calgon OL is not toxic to BA 101 and simultaneous separation of butanol from the fermentation broth appears commercially feasible.

### Example 2

### BA101 Fermentation with Various Adsorbent Additions

### Organism, Culture Maintenance, and Fermentation Conditions

C. beijerinckii BA 101 was used for these studies. Spores (200 μl) were heat shocked for 10 min. at 80° C. followed by cooling in an anaerobic chamber for 5 min. The culture was inoculated into 10 ml Tryptone-glucose-yeast extract (TGY) medium (in 50 ml screw capped pyrex bottle) and was incubated anaerobically for 12-14 h at 36±1° C.

The composition of the TGY media is as follows: Tryptone (30 g/L), Glucose (20 g/L), Yeast extract (10 g/L). Other nutrient media can be used. Useful nutrient media include those known to the art, such as P2. The nutrient media can optionally contain additives such as salts.

The composition of P2 media is as follows: Glucose (60-100 g/L) and Yeast extract (1-1.5 g/L). On cooling to 35° C. under oxygen-free nitrogen atmosphere, filter-sterilized P2 stock solutions [(buffer: KH₂PO₄, 50 gL⁻¹; K₂HPO₄, 50 gL⁻¹; Ammonium acetate, 220 gL⁻¹), (vitamin: Para-amino-benzoic acid, 0.1 gL⁻¹; Thiamin, 0.1 gL⁻¹; Biotin, 0.001 gL⁻¹), (mineral: MgSO₄.7H₂O, 20 gL⁻¹; MnSO₄.H₂O, 1 gL⁻¹; FeSO₄.7H₂0, 1 gL⁻¹; NaCl, 1 gL⁻¹)] were added.

### Batch Fermentation

These experiments were done in 100 mL batches in a 160 mL milk dilution bottle, placed inside an anaerobic chamber in an incubator maintained at 35° C. This media was prepared, autoclaved, and equilibrated in anaerobic conditions prior to inoculation. These bottles (batch reactors) were inoculated with the 10 mL □noculums of TGY extract with a large number of C. beijerinckii BA101 prepared as described in the previous paragraphs.

### Analysis of Fermentation

Glucose concentration was determined using a hexokinase and glucose-6-phosphate dehydrogenase (Sigma Chemicals, St. Louis, Mo., USA) coupled enzymatic assay. The analysis of the media was performed for the ABE and acids concentration using the GC analysis. The total amount of ABE produced and acids (acetic and butyric) were measured using a 6890 Hewlett-Packard gas chromatograph (Hewlett-Packard, Avondale, Pa.) equipped with a flame Ionization detector (FID) and 6 ft×2 mm glass column (10% CW-20M, 0.01% H₃PO₄, support 80/100 Chromosorb WAW). The measurement procedure was as follows:

Preparation of Acetone-Butanol-Ethanol standard: A) Standard solutions of acetone, butanol, and ethanol were prepared with distilled water (acetone 2 g/L, butanol 5 g/L, and ethanol 2 g/L). B) A standard solution (50 g/L) of internal standard (n-propanol) was prepared with distilled water. 1 ml of A and 0.1 ml of B were mixed. 1 μL of the mixture was injected into GC and the peak areas of acetone, butanol, ethanol and n-propanol were shown in the chromatogram. The order of the peaks is acetone, ethanol, n-propanol, butanol, Acetic acid, and Butyric acid.

Preparation of samples for GC analysis: Aliquots of samples were taken from the fermentor and centrifuged at 14,000 rpm for 3 min at 4° C. 25 μL of the internal standard was added to 250 μL of the supernatant and mixed. 1 μL of the mixture was injected into GC and the chromatogram displayed the individual ABE peak areas. The concentration of the acetone, butanol, or ethanol is calculated as follows:

From the peak areas, Response Factors (RF) for each peak was calculated as follows: acetone (RF)=(internal standard peak area/acetone peak area)/(wt of internal standard(5 g)/acetone wt(2 g)) Conc. in g/L(acetone)=(Wt of internal standard(5 g)×RF×Peak area of acetone)/(Peak area of internal standard)

### Adsorbent Addition

Four adsorbents were added to separate batch systems: Amberlite XAD 4, C18 (generally 50 μm particles with 60 A pore size), Zeolite 13× Molecular Sieve, and Orpheus Silicalite (40-50 mesh particle size bound with Alumina).

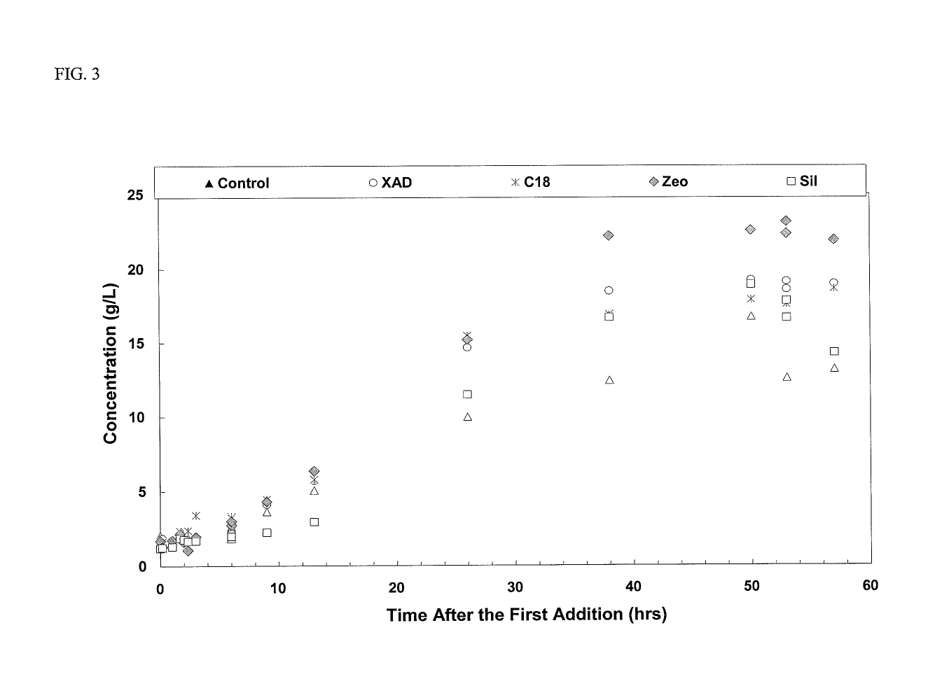

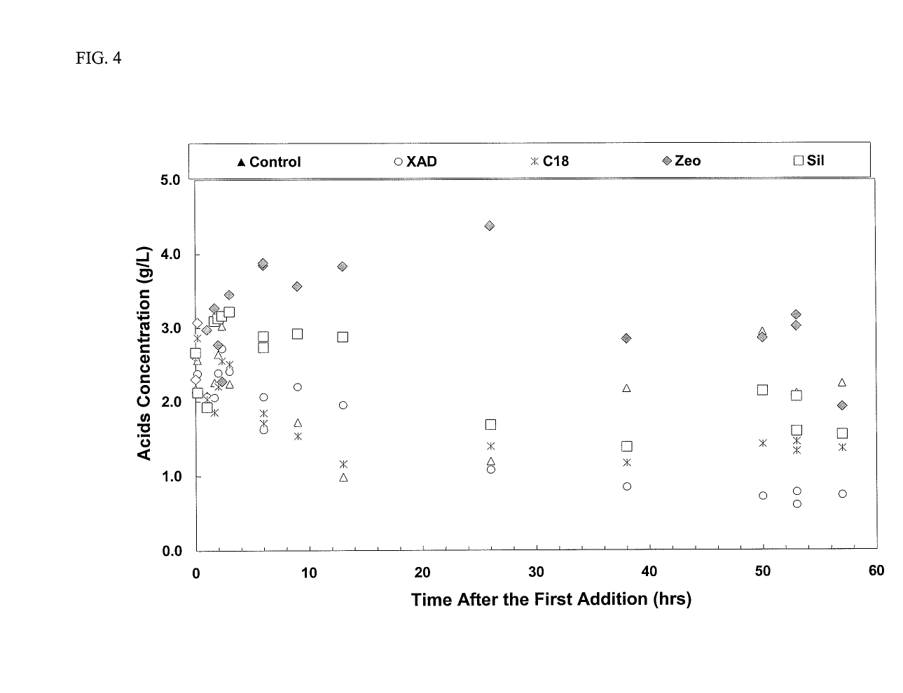

The adsorbents were added at 10, 12, 14 and 20 hours from the start of the fermentation. The concentration of acetone, butanol, and ethanol (ABE) in g/L and the concentration of intermediate acids (such as acetic and butyric acids) in g/L were analyzed as discussed above. The results are shown in FIG. 3 and FIG. 4.

Referring to FIG. 3 and FIG. 4, the solventogenic fermentation continued subsequent to the addition of the adsorbents, which furthers the proposition that the adsorbents are not toxic to Clostridium beijerinckii BA101.

Thus, the adsorbents are not toxic to BA 101 and simultaneous separation of butanol from the fermentation broth appears commercially feasible.

### Example 3

### Solvent Recovery from Fermentation Broth via a Continuous Expanded Bed Adsorption Process

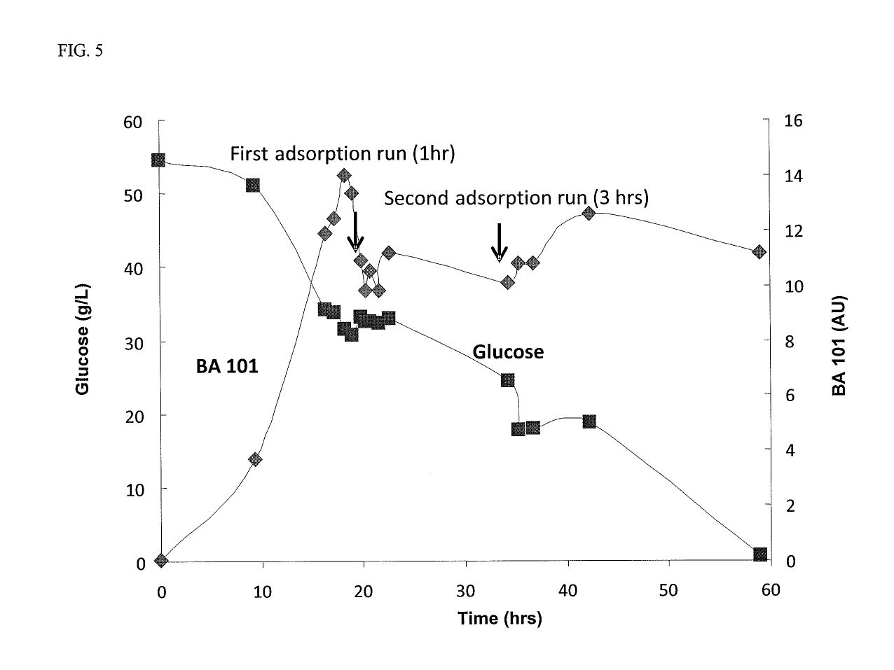

This pilot plant experiment for continuous production of ABE Fuel included fermentation of glucose, as described in above Experiments 1 and 2, with an expanded bed adsorption and thermal desorption process. The same reaction medium, organism, fermentation conditions, and analysis procedure of Examples 1 and 2 were adopted herein. OL Carbon was utilized as the adsorbent and was included in two 2 L vessels. The fermentor was a 10-Liter tank coupled to the adsorption bed unit. The fermentation reaction medium was circulated through a bottom-feed adsorbent bed, which was fluidized to eliminate any particulate plugging. The reaction medium was recycled to the fermentor subsequent to circulating through the adsorption bed unit. As indicated on FIG. 5, the reaction medium was fed to the adsorption bed after 10 hours of fermentation (for about an hour) and after 35 hours of fermentation (for about 3 hours). The concentration of glucose (g/L) and concentration of C. beijerinckii BA 101 (AU) were measured throughout the experiment. The concentration of C. beijerinckii BA 101 was measured using an A600 spectroscopy unit. After the adsorption phase, the bed was regenerated by passing CO₂ through the bed and heating the bed. The solvents desorbed were collected and analyzed. After the second adsorption phase, the fermentor was left alone to demonstrate continued activity of the bacteria and establish lack of toxicity to the system.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for separating a solvent from a reaction medium of a fermentative solventogenesis process, using Clostridium beijerinckii NCIMB 8052 or derivatives thereof, comprising:
contacting the reaction medium directly with an adsorbent that selectively adsorbs the solvent from the reaction medium;
separating the adsorbent/solvent adsorbate from the reaction medium; and
desorbing the solvent adsorbate from the adsorbent.

2. The method of claim 1, wherein the contacting of the reaction medium directly with the adsorbent occurs during at least a portion of the fermentation.

3. The method of claim 2, wherein the reaction medium contacting the adsorbent includes solventogenic organisms.

4. The method of claim 1, wherein the solvent comprises butanol, wherein the butanol concentration of the reaction medium is between 0.1% w/v and 2.0% w/v.

5. The method of claim 1, wherein the solvent comprises butanol, wherein the concentration of butanol in the reaction medium is between 0.5% w/v and 0.7% w/v.

6. The method of claim 1, wherein the concentration of solvent in the reaction medium is between about 1% w/v and 10% w/v.

7. The method of claim 1, wherein the concentration of solvent in the reaction medium is less than 12% w/v.

8. The method of claim 3, wherein the solventogenic microorganisms include Clostridium beijerinckii BA101.

9. The method of claim 1, wherein the solvents comprise one or more of acetone, butanol, and ethanol.

10. The method of claim 1, wherein contacting the reaction medium directly with an adsorbent occurs in a fermentor.

11. The method of claim 12, wherein contacting the reaction medium directly with the adsorbent is a continuous, countercurrent process.

12. The method of claim 1, wherein contacting the reaction medium directly with the adsorbent occurs in one or more separation units comprising a bed of adsorbent.

13. The method of claim 12, wherein the bed of adsorbent is an expanded bed.

14. The method of claim 12, wherein the one or more separation units operate in a swing-bed system.

15. The method of claim 13, wherein the expanded bed includes a magnetically stabilized fluid bed.

16. The method of claim 1, wherein the fermentative solventogenesis process operates at a pressure of about 550 mmHg.

17. The method of claim 1, wherein the fermentative solventogenesis process operates at a pressure between 450 mmHg and 650 mmHg.

18. The method of claim 1, wherein the fermentative solventogenesis process operates at a pressure less than 2 atm.

19. The method of claim 1, wherein the fermentative solventogenesis process operates at a temperature of about 37° C.

20. The method of claim 1, wherein the fermentative solventogenesis process operates at a temperature between 27° C. and 47° C.

21. The method of claim 1, wherein the desorbing the solvent adsorbate from the adsorbent step operates at a temperature of about 90° C.

22. The method of claim 1, wherein the desorbing the solvent adsorbate from the adsorbent step operates at a temperature between 70° C. and 160° C.

23. The method of claim 1, wherein the fermentative solventogenesis process operates at a temperature of about 37° C. and the desorbing the solvent adsorbate from the adsorbent step operates at a temperature around 80° C.

24. The method of claim 1, wherein desorbing the solvent adsorbate from the adsorbent includes thermal desorption facilitated with carbon dioxide.

25. The method of claim 1, wherein the fermentative solventogenesis process operates at a pH of about 4.8.

26. The method of claim 1, wherein the fermentative solventogenesis process operates at a pH between 4 and 7.

27. The method of claim 1, wherein the adsorbent includes one or more of silica, silicalite, bonded silica (C18), and end capped silica.

28. The method of claim 1, wherein the adsorbent includes one or more of carbon and activated carbon.

29. The method of claim 28, wherein the adsorbent is activated carbon and the activated carbon includes Calgon OL.

30. The method of claim 1, wherein the adsorbent includes one or more of alumina and functionalized alumina.

31. The method of claim 1, wherein the adsorbent includes zeolites.

32. The method of claim 1, wherein the adsorbent includes cation exchanged zeolites.

33. The method of claim 1, wherein the adsorbent includes polymers.

34. The method of claim 1, wherein the adsorbent includes hydrophobic adsorbents.

35. The method of claim 34, wherein the hydrophobic adsorbents include C18.

36. The method of claim 1, wherein the adsorbent includes XAD 4.
